(19) United States
(12) Patent Application Publication
(10) Pub. No.: US 2007/0199568 A1
(43) Pub. Date: Aug. 30, 2007

(54) NASAL DRUG DELIVERY

(75) Inventors: Colin Diekens, Towcaster (GB); Julia Kimbell, Chapel Hill, NC (US); Jeffry Schroeter, Cary, NC (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: BESPAK PLC (INCORPORATED IN THE UNITED KINGDOM), Bucks, MK12 5TS (UK)

(21) Appl. No.: 10/596,433

(22) PCT Filed: Dec. 15, 2004
(86) PCT No.: PCT/GB04/05252

§ 371 Date: Feb. 12, 2007

(30) Foreign Application Priority Data
Dec. 15, 2003 (GB) 0329041.8
Jan. 12, 2004 (GB) 0400586.4

(51) Int. Cl. A61M 15/00; A61M 15/08; B65D 83/06

(52) U.S. Cl. 128/207.18; 128/203.15

(57) ABSTRACT

A device and method is provided for delivering an active material or drug in particular form to a targeted region of the nasal passage, such as the turbinates. The device includes a nozzle outlet (28) for the material, supported for example in an outer nozzle (20) which fits the nostril so as not to disrupt non-turbulent flow into the nostril. The particles are released from the outlet (28) into the non-turbulent flow at a similar velocity, in order to follow the airstream. Thus it is possible to predict their likely path, such that preferred release points can be calculated.

This invention relates to the delivery of drugs to a target region of the nasal passage, such as the turbinate region. These can include pain management drugs, vaccines, biologics and hormones, amongst others.

Nasal drug delivery devices are available which deliver a drug or active material to the nasal passages, for example in the form of a spray of particles. If it is a requirement that the drug is absorbed into the bloodstream, it is preferable to deposit the particles in the turbinate region of the nasal passage, or on the surrounding tissue. The turbinates are an ideal deposition site for systemic drugs due to their highly vascularized nature. The tissue surrounding this area is also a good deposition site, having the same cell type.

Available devices usually propel drug particles into the nose from a nozzle inserted into the nostril. In such cases, it is difficult to predict where the particles will deposit. In practice, it has been found that a large fraction of particles fail to penetrate the nasal valve, and some are likely to deposit in the olfactory region. This may be undesirable for the delivery of certain drugs such as vaccines, where deposition in the olfactory region may provide a direct route to the brain, bypassing the blood-brain barrier which protects the brain from foreign material.

Alternatively, it is sometimes considered advantageous to target the olfactory region, since this may offer a better route for drugs which are beneficially delivered to the brain, such as pain relievers or drugs for conditions affecting brain function, such as Parkinson's disease.

This invention seeks to increase deposition in the region of the nose that has been identified as a target. The invention also seeks to reduce undesirable effects associated with deposition in other regions.

According to one aspect of the present invention there is provided a device for delivering an active material to a target region of a nasal passage, comprising delivery means having an outlet for the material, and support means for supporting the delivery means with the outlet at a predetermined location in the nostril, the device being arranged such that gas flow into the nostril is substantially stable and non-turbulent.

By providing the outlet in a stable and non-turbulent flow, such as a laminar flow, particles released therefrom will follow the airstream. Therefore the likely deposition of the particles can be predicted using fluid dynamics. In particular, a computer model of the nasal cavity can be used together with computational fluid dynamics methods to calculate particle behaviour. Thus, it is possible to determine a preferred location in the nostril for the outlet, from which most particles will deposit in a target region such as the turbinates.

It has been found that airflow into the nostril is stable and non-turbulent for constant airflow typical of human inhalation rates. Thus, the device may be arranged such that inhalation gas flow into the nostril is substantially unimpeded.

For example, the support means may comprise a nostril engaging portion such as an outer nozzle through which the subject may inhale, and the delivery means may comprise an inner delivery nozzle. The outer nozzle may be arranged to fit within the nostril and to be substantially coextensive with the nostril in a direction substantially perpendicular to the direction of flow.

The delivery nozzle may have a gas inlet within the outer nozzle, and the outlet of the delivery nozzle may be at or adjacent the outlet of the outer nozzle. The active material may be released into the delivery nozzle and entrained by the flow therethrough. By arranging the device such that air resistance through the delivery nozzle is minimised, the flow speed at the outlet of the outer nozzle will be substantially the same as that at the outlet of the delivery nozzle.

Alternatively, the delivery means may be small and have an aerodynamic configuration such that it does not impede the laminar flow, and such that the particles emitted from the outlet are entrained by the flow. For example, it may be a “needle” shaped head for emitting particles by electrostatic means. It may be supported in the nostril either within an outer nozzle, or by other support means.

The device may include a housing for containing the particles of active material, and a delivery passage communicating with the delivery means or nozzle. The device conveniently includes means for providing the particles as an aerosol. This may be a soft mist device such as a nebulizing head.

As an alternative to using inhalation to provide the laminar flow, the device may include means for propelling gas into the nostril at a suitable velocity, and may also include means for propelling the particles from the outlet at substantially the same velocity. For example, the device may have an outer nozzle and a delivery nozzle as already described, with the gas flow therethrough being provided by a “bellows” type plunger, such as that shown in our co-pending application, WO 02/30500. Alternatively the particles may be entrained in a separate delivery gas flow.

Suitable flow rates for providing a non-turbulent airflow have been found to be from about 1 litre/min to about 30 litres/min. At such flow rates, the velocity of the delivery gas flow, or of the particles, should be within about +/−20% of that of the flow into the nostril in order to follow the airstream.

The particle size influences how closely the particles will follow the airstreams. For example, where the flow undergoes rapid changes of direction, such as when moving round a sharp corner, larger particles will change direction more slowly, leading to such particles becoming entrained in different airstreams, or depositing on the walls of the nasal cavity. Whilst smaller particles are more likely to stay in the airstreams, they are less likely to deposit in the nasal cavity, and may be carried through to the throat or lungs. It has been found generally that the particles preferably have an aerodynamic diameter from about 10 μm to about 20 μm.

For deposition in the turbinate and surrounding regions, the particles preferably have an aerodynamic diameter from about 7.5 μm to about 50 μm, or more preferably about 30 μm, and still more preferably from about 10 μm to about 20 μm. For olfactory deposition, the size range is preferably about 7.5 μm to about 20 μm, and more preferably from about 10 μm 15 μm.

Since particles of different sizes have different deposition behaviour, the predetermined location may be different depending upon the particle size of the active material. For example, for turbinate deposition, the predetermined location may be nearer the base of the nose for larger particles, and nearer the tip of the nose for smaller particles.

Preferably the device includes a guide arranged to guide the support means or outer nozzle into a predetermined orientation with respect to the nostril, such that the outlet of the delivery means is positioned at the required predetermined location. For example, the guide may include a base plate mounting a further outer nozzle for engaging the other nostril, and a frame member for passing over the bridge of the nose to ensure the nozzles are inserted upright. The base plate also serves to abut the nostril such that the nozzle outlet is inserted a predetermined distance into the nostril.

The device may be arranged such that it is possible to deliver the active material selectively through either nostril. Thus, there may be support means and delivery means corresponding to each respective nostril, or the support means and delivery means may be movable between positions corresponding to respective nostrils.

For example, where there is a further outer nozzle, this may also be provided with a delivery nozzle, and the housing and delivery passage may be attachable to either of the outer nozzles. Alternatively, a single outer nozzle mounted on a guide may be movable between positions corresponding to each respective nostril.

Thus from another aspect, the invention provides a device for delivering a substance selectively to either nostril comprising: a guide arranged to cooperate with the nose, a pair of delivery stations arranged to correspond with each respective nostril, and substance delivery means positionable at either of the delivery stations.

From yet another aspect, the invention provides a method of delivering an active material to a target region of the nasal passage, comprising delivering the material from a predetermined location in the nostril in a substantially non-turbulent gas flow which surrounds the location.

The invention will now be described by way of example with reference to the accompanying drawings, in which:—

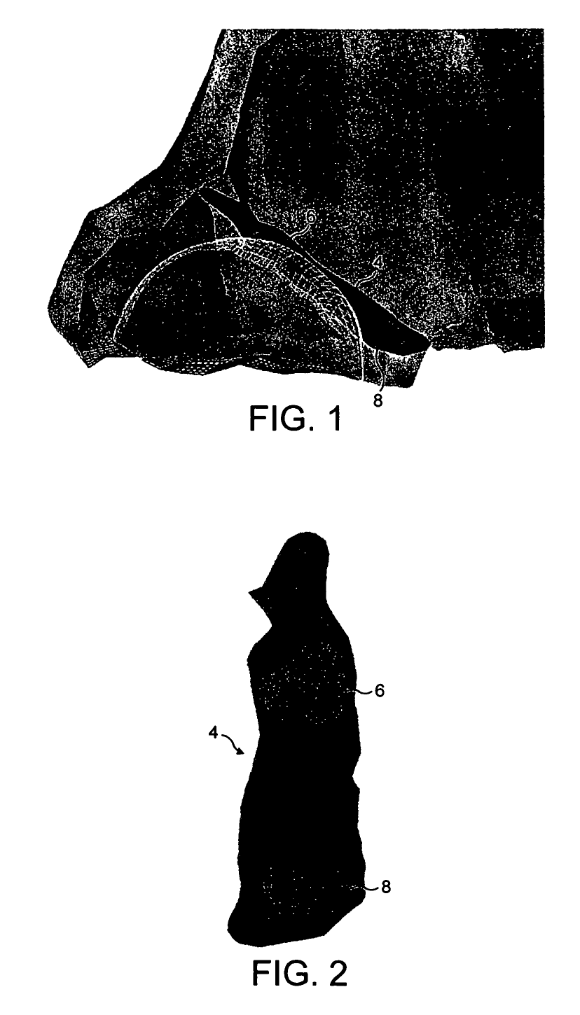

FIG. 1 is a schematic perspective view of a nostril;

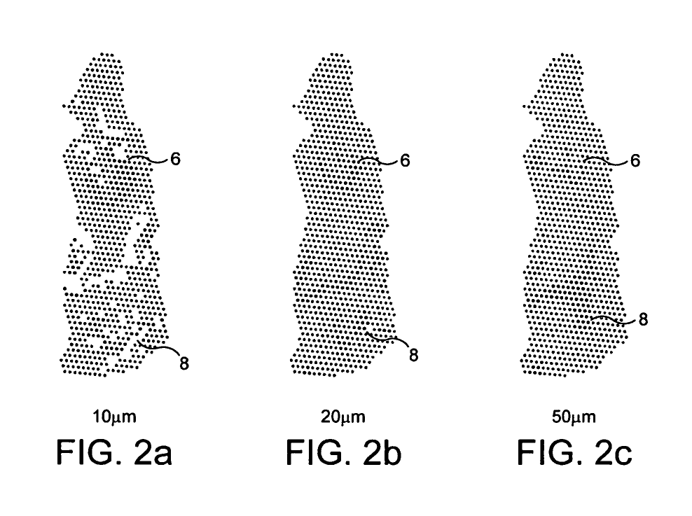

FIG. 2 is a cross sectional view of a ‘release plane’ for the nostril of FIG. 1;

FIGS. 2a, b and c are further cross-sectional views of the release plane;

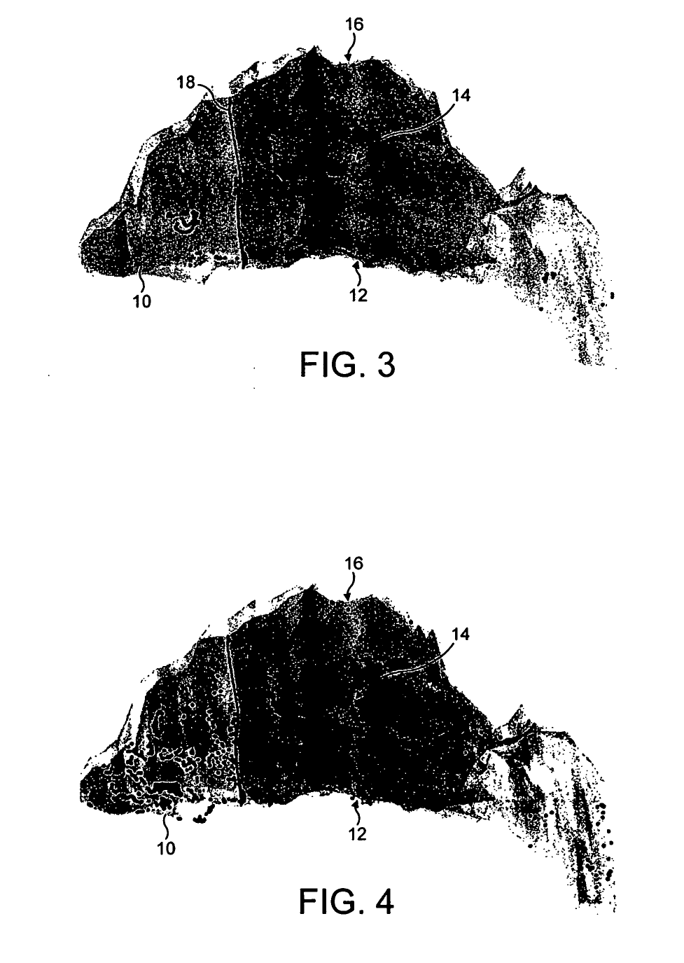

FIG. 3 is a side view of a nasal cavity showing particle deposition;

FIG. 4 is another side view of a nasal cavity showing particle deposition according to the prior art;

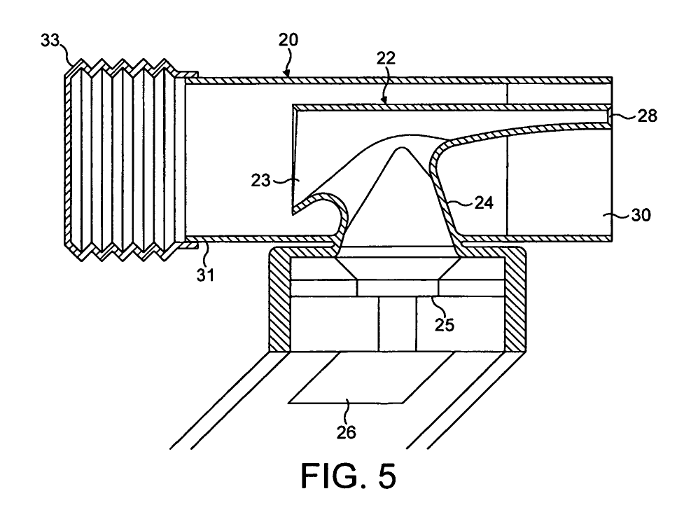

FIG. 5 is a cross sectional side view of a nozzle for a device according to one embodiment of the invention;

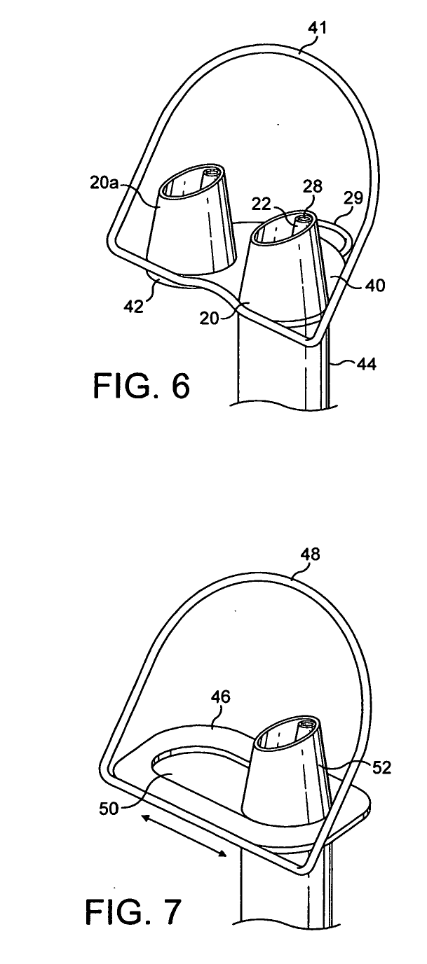

FIG. 6 is a perspective view of a device according to another embodiment of the invention,

FIG. 7 in a perspective view of a further device according to an embodiment of the invention, and

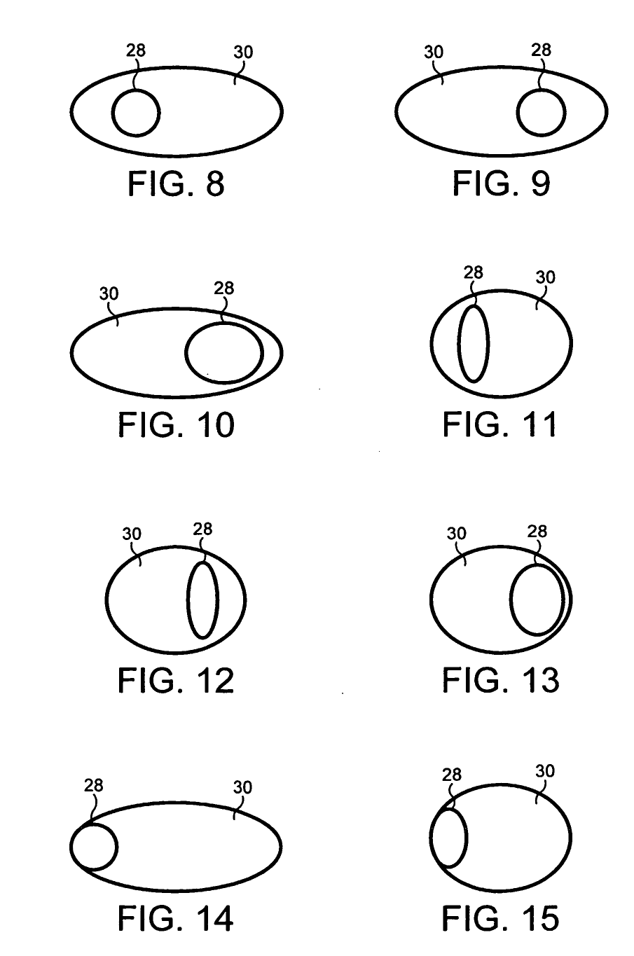

FIG. 8 to 15 are diagrammatic cross-sectional views of nozzles according to various embodiments of the invention.

Referring to FIGS. 1 and 2, the cross-section shown in FIG. 2 is a plane 4 in the nostril forming about a 45° angle with the horizontal, tilted towards the nasal passages, and extending about 1 cm into the nose from a point near the centre of the nostril opening 3. This is an example of a typical release region, where particles of active material can be released from a nozzle inserted into the nostril through the nostril opening 3. It has been found that particles released from certain locations on this plane have a much greater likelihood of a depositing in the turbinate region than those released uniformly across the plane.

Referring to FIGS. 3 and 4, the nasal passage comprises the following parts. The nasal vestibule 10 is in the area directly inside each nostril opening 3. The turbinate region includes the inferior turbinate 12, the middle turbinate 14, and the superior turbinate 16, which includes the olfactory region. A narrowing of the air passages between the vestibule 10 and the turbinates 12, 14 and 16 is known as the nasal valve 18. The turbinate region 12, 14 and 16 is lined with respiratory epithelium cells, and has a plentiful supply of blood vessels. This tissue is a major target for drug delivery, allowing a quick route into the blood supply.

FIG. 3 shows the results of a simulation of particles released from the overlapping circular areas 8 in the lower part of the plane 4, and from the overlapping circular areas 6 in the upper portion of the plane 4. The dark patches represent the positions of particle deposition. It can be seen that a large proportion of the particles were predicted to deposit in the lower inferior turbinates 12 and the lower middle turbinates 14. Furthermore, it was found that no particles released from these areas 6 and 8 deposited in the olfactory region of the nose.

In contrast, a simulation of particles released over the entire area of the plane 4, shown in FIG. 4, shows that a large proportion of the particles deposit in the nasal vestibule 10 and fail to reach the turbinates. Also, some particles deposit in the olfactory region in the superior turbinate 16. The number of particles depositing in the target region was found to be 2.5 times higher when the particles were released from the specific location 6 and 8.

In FIGS. 2a to c, the darker regions show the release points from which particles were predicted to deposit in the turbinates, for particles of different sizes. In this example, the particles are assumed to be passively released in the presence of steady-state inspiratory airflow at a total volumetric rate of 15 L/min. No account is taken if any disruption to the airflow by the presence of a delivery device. It can be seen that the release points tend to cluster around areas corresponding to the upper or ventral region 6, and the lower or dorsal region 8 in FIG. 2 for all particle sizes. However, the favoured release points for smaller particles (eg around 10 μm) were those in the ventral area 6, and for larger particles (eg above about 20 μm) were those in the dorsal area 8.

A suitable device for delivering particles to these locations is shown schematically in FIG. 5. The device comprises an outer nozzle 20, an outlet end 30 of which is configured for insertion in the nostril. The opposite end 31 is attached to a “bellows” 33 for providing gas flow into the nozzle 20. The outlet 30 of the outer nozzle 20 is sized and configured so as to substantially fill the nostril, such that airflow from the outlet 30 into the nostril will have a substantially uniform profile.

The outer nozzle 20 acts as a support means for an inner delivery nozzle 22, mounted therein by a hollow stem 24. The stem 24 provides communication between the interior of the delivery nozzle 22 and a housing 26 for containing the active material, via a dosing head 25. An inlet 23 of the delivery nozzle 22 acts as a flow splitter, with both the delivery nozzle 22 and the stem 24 being configured so as substantially not to disrupt non-turbulent flow in the outer nozzle 20. The outlet 28 of the delivery nozzle 22 is positioned at a predetermined location within the outlet 30 of the outer nozzle 20.

When the outer nozzle 20 is inserted into a nostril, the outlet 28 of the delivery nozzle 22 is supported at the required location in the nostril for delivery of the active material. For example, the outlet 30 of the nozzle 20 may lie on a plane such as the plane 4 shown in FIG. 2. The outlet 28 of the delivery nozzle may be located to correspond with the areas 6 or 8 within the plane 4.

When the bellows 33 is compressed, a non-turbulent airflow is created through the outer nozzle 20, which splits such that part of the flow enters the delivery nozzle 22. The airflow through the delivery nozzle 22 draws air from the housing 26 through the stem 24 into the delivery nozzle 22, entraining particles of the active material from the housing 26. The active material may be provided in the form of an aerosol created using a soft mist device such as a nebulizing head (not shown).

Alternatively, the bellows 33 may be omitted. In this case, the non-turbulent flow may be provided in the outer nozzle by the user inhaling.

A nozzle configuration such as that in FIG. 5 may be used with a guide as shown in the device of FIG. 6. The outer nozzle 20 is mounted on a base plate 40. A further nozzle 20a is mounted on the base plate 40 adjacent the first nozzle 20 such that the nozzles may be inserted into the nostrils of a user, with the base plate abutting against the end of the nose.

A wire frame member 41 extends from each side of the lower edge 44 of the base plate for resting on the bridge of the nose when the device is in position. This ensures the nozzles cannot be inserted upside-down, and thus that the outlet 28 of the delivery nozzle 22 is correctly positioned relative to the nostril.

With such a device, it is possible that either nozzle 20a may be used to administer the active material selectively to either nostril.

In this case, the nozzles 20, 20a each have an outlet port comprising an aperture in the base plate 40, which is attachable to either of a shut-off plate 42 or the main body 44 of the device (partly shown) as described above. This may be achieved by a handle 29 which may be operated to switch around the main body 44 and the shut-off plate 42 so as to align the main body 44 of the device with the nozzle selected for delivery.

FIG. 7 shows an alternative guide for allowing selection of the delivery nostril. In this example, the device has a base plate 46 and a frame member 48 similar to those (40, 41) of FIG. 7. However, the base plate includes a channel 50 for receiving a single outer nozzle 52 which is connected to the main body of the device (not shown). The outer nozzle 52 can be positioned at either end of the channel 50 by sliding it along the channel, so as to correspond with either nostril.

Each of FIGS. 8 to 15 show examples of how the outlet 28 of the delivery nozzle may be positioned relative to the outlet 30 of the outer nozzle.

In particular, FIG. 8 shows an outer nozzle having an oval shaped outlet 30, in cross-section. A circular outlet 28 of a delivery nozzle is positioned with its centre a little less than one third of the length of the outer nozzle outlet 30 away from the end which is to be aligned with the tip of the nose. It has a diameter a little over half of the width of the outer nozzle outlet 30 at that position. This configuration aims to target the turbinate region of the nose.

An alternative configuration for targeting the turbinates is shown in FIG. 9 This is similar to that of FIG. 8 but is reversed such that the delivery nozzle outlet 28 is nearer the other end of the outer nozzle outlet 30 (i.e. the end which is to be aligned with the base of the nose). Whilst either of these configurations may be used for particles over the preferred range of sizes, that of FIG. 8 favours smaller particles, and that of FIG. 9 larger particles.

FIGS. 11 and 12 are equivalent to FIGS. 8 and 9, but are for use with a more rounded nostril shape. Thus the shapes of the outlet 30 of the outer nozzle and of the delivery nozzle outlet 28 are both shorter and wider, but have the same respective positions.

FIG. 10 is an example of a configuration aimed to target the inferior turbinates and surrounding tissue. In this embodiment, the delivery nozzle outlet 28 is circular, and positioned with its centre about three quarters of the length of the outer nozzle outlet 30 away from the end to be aligned with the tip of the nose. Its diameter is about three-quarters of the width of the outer nozzle outlet 30 at that point. FIG. 13 shows an equivalent configuration for a more rounded nostril, with the shapes of the outlets 28 and 30 shorter and wider, but in the same respective positions.

In FIGS. 14 and 15, configurations for targeting the olfactory region are shown for more oval and more rounded nostrils respectively. In FIG. 14, the delivery outlet 28 is circular, and has a diameter about one quarter of the length of the outer nozzle outlet 30. It is positioned against the end of the outlet 30 which is to be aligned with the tip of the nose. In FIG. 15, the shapes are shorter and wider.

Drugs for nasal delivery are commonly provided in aerosol form. The aerodynamic particle size or “aerodynamic diameter” is a term used in aerosol physics to provide a particle size definition that relates directly to how a particle behaves in a fluid such as air. For non-spherical particles, clearly the term “diameter” is not applicable. For example, the particle may be a flake or a fibre. Moreover particles having the same diameter which are composed of different chemical compounds may have different densities. Thus, the aerodynamic diameter is the equivalent diameter of a spherical particle having a density of 1 g per cubic centimetre that has the same inertial properties (i.e. terminal settling velocity) in the fluid as the particle of interest. An inertial sampling device such as a cascade impactor can be used for particle sizing. Such a sampling device can be used to determine the aerodynamic diameter.

The particles containing the active material may be mixed with particles of a carrier material. For example the carrier material particles may be much larger, for facilitating handling of the mixture of the active and carrier material particles, such as pouring into a delivery device. The larger particles of carrier material may deposit in the nasal vestibule upon delivery, with the particles of active material continuing into the target region.

Furthermore, any reference herein to particles of an active material includes particles containing both an active material and a carrier material. Thus, a given range of particle sizes may include particles wholly comprised of active material and particles comprised of active material and carrier material. The carrier material may be any pharmaceutically acceptable material, such as lactose or calcium carbonate.

1. A device for delivering an active material to a target region of a nasal passage, comprising delivery means having an outlet for the material, and support means for supporting the delivery means with the outlet at a predetermined location in the nostril, the device being arranged such that gas flow into the nostril from said delivery means is substantially stable and non-turbulent.

2. A device as claimed in claim 1, in which the device is arranged such that inhalation gas flow into the nostril is substantially unimpeded.

3. A device is claimed in claim 1, in which the device is arranged such that the gas flow into the nostril substantially surrounds the outlet.

4. A device is claimed in claim 1, further comprising a guide arranged to guide the support means into a predetermined orientation with respect to the nostril.

5. A device as claimed in claim 1, in which the delivery means comprises a delivery nozzle.

6. A device as claimed in claim 1, in which the support means comprises an outer nozzle.

7. A device as claimed in claim 6, in which the outer nozzle is arranged to fit within the nostril and to substantially coextensive with the nostril in a direction substantially perpendicular to the direction of flow.

8. A device as claimed in claim 4, further comprising an outer nozzle, and in which the guide comprises abutment means mounted on the outer nozzle for abutting the outlet of the nostril.

9. A device as claimed in claim 4, in which the guide comprises means for cooperating with the other nostril.

10. A device as claimed in claim 9, in which the means for cooperating with the other nostril comprises a plurality of outer nozzles, and the abutment means comprises a base on which the outer nozzles are mounted.

11. A device as claimed in claim 10, in which the support means comprises either of the outer nozzles.

12. A device as claimed in claim 4, in which the support means is movable with respect to the guide between positions corresponding to each nostril respectively.

13. A device as claimed in claim 12 in which the guide comprises a member arranged to cooperate with the nose such that the device may be positioned in one orientation only.

14. A device as claimed in claim 5, further comprising an outer nozzle, and in which the inlet of the delivery nozzle is within the outer nozzle, and the outlet of the delivery nozzle is at or adjacent the outlet of the outer nozzle.

15. A device as claimed in claim 14, comprising a housing for containing particles of active material, and a delivering passage communicating with the delivery nozzle.

16. A device as claimed in claim 1, further comprising aerosol means for providing particles of active material as an aerosol mist.

17. A device as claimed in claim 1, comprising gas propulsion means for propelling gas into the nostril at a flow rate which produces the substantially non-turbulent flow.

18. A device as claimed in claim 17, in which the gas propulsion means is arranged to propel the gas flow rate from about 1 litre/min to about 30 litres/min.

19. A device as claimed in claim 1, comprising particle propulsion means for propelling particles of active material from the outlet at a velocity substantially matching that of non-turbulent flow.

20. A device as claimed in claim 19, in which the particle propulsion means is arranged to entrain the particles in a delivery gas flow, the delivery gas flow having a velocity about +/−20% of that of the non-turbulent flow.

21. A device for delivering a substance selectively to either nostril comprising: a guide arranged to cooperate with the nose, a pair of delivery stations arranged to correspond with each respective nostril, and substance delivery means positionable at either of the delivery stations.

22. A method of delivering an active material to a target region of the nasal passage, comprising delivering the material from a predetermined location in the nostril in a substantially non-turbulent gas flow.

23. A method as claimed in 22, comprising delivering the particles at a velocity substantially matching that of the gas flow.

24. A method as claimed in claim 22, comprising providing the non-turbulent gas flow in the nasal passage.

25. A method as claimed in claim 22, in which the predetermined location is surrounded by the substantially non-turbulent gas flow.

26. A method as claimed in claim 22, comprising providing the material in the form of particles having aerodynamic diameter from about 7.5 μm to about 30 μm.

27. A method as claimed in claim 26, in which the aerodynamic diameter is from about 10 μm to about 20 μm.

28. A method as claimed in claim 27, in which the aerodynamic diameter is from about 10 μm to about 15 μm.

29. A method as claimed in claim 26, in which the aerodynamic diameter is from about 7.5 μm to about 20 μm.

30. A method as claimed in claim 22, in which the target region is the olfactory region, and in which the predetermined location is at or adjacent the tip of the nose.

31. A method as claimed in claim 22, in which the target region is the turbinate region, and in which the predetermined location is spaced from the tip of the nose, and contained within an area which is closer to the tip of the nose than the base of the nose.

32. A method as claimed in claim 22, in which the target region is the turbinate region, and in which the predetermined location is spaced from the base of the nose, and is contained within an area which is closer to the base of the nose than the tip of the nose.

33. (canceled)

34. (canceled)
